(19) United States
(12) Patent Application Publication
(10) Pub. No.: US 2025/0163416 A1
(43) Pub. Date: May 22, 2025

(54) ACTIVATION OF NONCODING HOST GENE LOCI

(71) Applicant: The University Court of the University of Edinburgh, Edinburgh (GB)

(72) Inventors: Andrew Baker, Edinburgh (GB); Francesca Vacante, Edinburgh (GB)

(21) Appl. No.: 18/874,722

(22) PCT Filed: Jun. 12, 2023
(86) PCT No.: PCT/GB2023/051524

§ 371 Date: Dec. 13, 2024

(30) Foreign Application Priority Data
Jun. 13, 2022 (GB) 2208575.7

(51) Int. Cl. C12N 15/113; A61K 48/00; C12N 9/22; C12N 15/11; C12N 15/86

(52) U.S. Cl. CPC C12N 15/113; A61K 48/005; C12N 9/22; C12N 15/111; C12N 15/86; C07K 2319/71; C12N 2310/141; C12N 2310/20; C12N 2710/10043; C12N 2740/15043

(57) ABSTRACT

Disclosed is a construct for use in a method of transcriptional activation of a complex genomic locus. The construct comprises a single guide RNA (sgRNA) binding a regulatory element of the said complex genomic locus, and a deactivated Cas. The construct may be introduced into cells including the genomic locus of interest in order to activate the regulatory element to transcribe multiple transcripts within said complex genomic locus.

### FIELD

The present disclosure relates to methods of activating one or more complex genomic loci and compositions thereof.

### BACKGROUND

Different genomic entities, such as transcriptional isoforms of the same gene and noncoding RNAs belonging to the same locus, can synergistically regulate important cellular function(s). This is particularly important in the case where genes or noncoding RNA molecules are downregulated in pathological settings or during cell differentiation programs¹, for example. Therefore, global activation of the whole set of transcripts may be necessary to restore physiological cell function, maintain cell identity or to regulate cell differentiation. The expression level of noncoding RNAs, such as long noncoding RNA (lncRNA) and microRNA (miRNA), is a key factor regulating the physiological and pathological cellular states. In particular, the modulation of the expression of noncoding RNA molecules can influence cellular processes and cellular responses to pathological stimuli²⁴. Complex noncoding RNA loci can be described as genomic regions that comprise multiple noncoding RNAs, which share portions of transcribed regions. Although each of the noncoding RNA can have synergistic or independent function, noncoding RNAs belonging to the same complex locus can be regulated by the same functional regions (promoters or enhancers)⁵. In some cases, the expression of complex genomic loci, such as host gene's lncRNAs and co-localised microRNAs, can be regulated by multiple promoters within the same locus.

Exogenous overexpression of transcripts, such as to therapeutically modulate pathological states, can be achieved by single overexpression of each of the components of a complex locus⁶. However, identification of the correct individual components that would achieve the desired cellular effect is challenging. For instance, the presence of multiple promoters in a complex locus makes it difficult to identify transcripts existing in the same locus, and noncoding molecules (such as lncRNA) can have enhancer activity which cannot be studied at a transcriptional level⁷. In addition, some of the transcriptional isoforms might not be entirely annotated and this would limit the study of transcriptional isoforms (as in the case of several lncRNAs)³. Current strategies, such as plasmids, viral vectors, short harpin RNAs or mimics, allow the individual overexpression of specific transcripts or microRNAs. However, this is not possible when i) the transcript size exceeds the vector limit, ii) the locus of interest includes multiple transcripts or iii) the structure of the locus in not well annotated.

The overexpression of transcripts or primary transcripts (such as the full length of pri-microRNA transcripts) has been previously explored⁹. This has been achieved by cloning a specific transcript sequence into expression plasmids or viral vectors in human cells or mouse models. This strategy has allowed the study of transcript-specific function but major limitations are associated with it: i) the annotated sequences might not reflect the accurate sequences of transcripts expressed by complex loci, due to incomplete characterisation of the loci or mis annotation of their transcripts, ii) it is difficult (and in some cases not feasible) to overexpress simultaneously all the isoforms belonging to the same locus, iii) viral vectors, such as lentiviruses or adeno-associated viruses (AAVs), cannot be used for the overexpression of large transcripts due to size limits¹⁰. These factors can be very important when considering a pathological context where the expression of multiple transcripts or noncoding molecules is required for therapeutic intervention.

The present disclosure provides a novel method of activating complex genomic loci, which overcomes one or more limitations of existing techniques, by globally enhancing the transcription of an entire transcriptional set.

### SUMMARY

The present disclosure is based in part on studies using two exemplary complex lncRNAs, CARMN and MIR503HG. Both host genes have been shown to be downregulated in different pathological contexts involving primary vascular cells. Therefore, the activation of their expression can be important to maintain vascular cell identity. The technology, as described and taught herein, was developed to activate one or more complex genomic loci in different cell types (primary cultures or cell lines) and in vivo models. The complex genomic loci that can be activated may include multi-transcript coding genes, noncoding RNA molecules, such as long noncoding RNAs and microRNAs, where the genes sharing the same regulatory regions. Surprisingly, the present inventors have demonstrated a method of globally activating an entire transcriptional set by activating a main regulatory element of a complex genomic locus, which overcomes the need of identifying the individual components of the complex genomic locus and/or overexpressing each of the individual transcripts of the complex genomic locus.

A method through which global transcriptional enhancement of a locus can be achieved is by activating the promoter (or enhancer region) of a complex genomic locus using CRISPRa technology, a variant of the canonical CRISPR/Cas9 known in the art. CRISPRa utilises deactivated Cas endonuclease (such as deactivated Cas9 (dCas9)), which recognises and binds a specific promoter/enhancer sequence which is complimentary to the single guide RNA (sgRNA). The binding of the complex, comprising sgRNA and dCas9, will favour the recruitment of transcriptional activators and the expression of the locus.

In a first aspect, there is provided a construct for use in a method of transcriptional activation of a complex genomic locus. The construct comprises a single guide RNA (sgRNA) binding a regulatory element of the said complex genomic locus, and a deactivated Cas. The construct is introduced into cells including the genomic locus of interest in order to activate the regulatory element to transcribe multiple transcripts within said complex genomic locus. Based on the evidence provided herein, the inventors have surprisingly found that they were able to activate the entire subset of transcripts expressed by the CARMN and MIR503HG loci, by only targeting a single promoter. Thus, the term “multiple transcripts” in the context of the present invention is understood to meen at least 50%, 60%, 70%, 80%, 90%, 95%, or substantially all (i.e. 100%) of the transcripts in any given complex genomic locus. Considering that each genomic locus can encode for a variable number of transcripts, this method allows to activate the transcription of the whole subset of transcripts included in the locus of interest.

A complex genomic locus as used herein refers to a genomic region in which multiple genes share transcribed regions. The multiple noncoding RNAs of a complex genomic locus typically share one or more portions of the transcribed regions, such as exons. Although each of the noncoding RNA can have synergistic or independent function, noncoding RNAs belonging to the same complex locus can be regulated by the same functional regions, such as promoters or enhancers. The genes of the said complex genomic locus may include protein coding genes, noncoding genes, multi-transcript coding genes, genes sharing the same regulatory regions (e.g. promoter(s) and/or enhancer(s)), or a combination thereof. Noncoding genes or noncoding DNA refer to regions within the genome that do not encode protein sequences when transcribed. Noncoding genes are considered to be an important regulator of various cellular functions, such as RNA maturation, RNA transport, chromatin remodelling, and transcriptional activation and/or repression programmes, for example. The noncoding genes may encode noncoding RNAs, such as long noncoding RNAs and microRNAs. Long noncoding RNAs (lncRNAs) are RNA molecules that are typically greater than 200 nucleotides in length and have low protein-coding potential. MicroRNAs (miRNAs) are short RNA molecules found within eukaryotic cells and typically comprises 20-25 nucleotides in length.

In one embodiment, the complex genomic locus may comprise one or more noncoding gene(s), multi-transcript noncoding gene(s), or a combination thereof. In some embodiments, the noncoding gene(s) comprise long non-coding RNA, microRNA, pri-microRNA, siRNA, piRNA, snoRNA, snRNA, exRNA, scaRNA or a combination thereof. In a preferred embodiment, the noncoding RNA comprises long non-coding RNA, microRNA and/or pri-microRNA, or a combination thereof. In some embodiments, the complex genomic locus may comprise noncoding genes, multi-transcript coding genes, or a combination thereof. In some embodiments, the complex genomic locus may optionally comprise one or more protein coding genes.

The term “regulatory element” is intended to include promoters, enhancers, and other expression control elements. Such regulatory elements are described, for example, in Goeddel, GENE EXPRESSION TECHNOLOGY: METHODS IN ENZYMOLOGY 185, Academic Press, San Diego, Calif. (1990). The regulatory element(s) allows for the expression of the nucleotide sequence, such as in a host cell when the vector is introduced into the host cell. The term “regulatory element” may also be referred to as a “main regulatory element”, a “main promoter” or a “main enhancer”, which refers to a common promoter, enhancer, or other expression control element capable of transcriptionally activating an entire complex genomic locus. Transcriptional activation of an entire complex genomic locus typically results in transcription of several noncoding genes, as well as protein coding genes in some instances. In one embodiment, the main regulatory element as used herein comprises a promoter or an enhancer. In a preferred embodiment, the main regulatory element as used herein comprises a promoter. In some embodiments, the main regulatory element comprises one or more promoters or enhancers. Regulatory elements include those that direct constitutive expression of a nucleotide sequence in many types of host cell and those that direct expression of the nucleotide sequence only in certain host cells (e.g., tissue-specific regulatory sequences). In a preferred embodiment, the regulatory element comprises a promoter or an enhancer. A tissue-specific promoter may direct expression primarily in a desired tissue of interest, such as muscle, neuron, bone, skin, blood, specific organs (e.g. heart, liver), or particular cell types (e.g. smooth muscle cells (SMCs), endothelial cells). Regulatory elements may also direct expression in a temporal-dependent manner, such as in a cell-cycle dependent or developmental stage-dependent manner, which may or may not also be tissue or cell-type specific.

The construct as disclosed herein comprises nucleotides that encode the sgRNA designed to target a regulatory element, such as a promoter or an enhancer, of the complex genomic locus of interest, a deactivated Cas protein and one or more regulatory elements required for the expression of the sgRNA and the deactivated Cas protein. In a preferred embodiment, the deactivated Cas protein is deactivated Cas9. In certain embodiments, the Cas protein may comprise any other suitable endonuclease with a DNA-binding activity, but lacks the ability to cleave DNA, in order to transcriptionally activate the complex genomic locus of interest by directing the necessary components to the appropriate locus. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Csy4, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof or modified versions thereof, wherein the Cas protein retains its DNA-binding activity but lacks the ability to cleave DNA.

The inventors have identified that activating the main regulatory element of a complex genomic locus, such as an upstream promoter of the individual components of a complex genomic locus, is sufficient to globally activate transcription of the individual components of the said complex genomic locus. An upstream promoter refers to a promoter that lies distal to the transcription start site of a gene of interest, which may typically be found −2 kbp, −1 kbp, −500 bp. −300 bp, −250 bp, −200 bp, −150 bp or −100 bp to 0 bp from the transcription start site. sgRNA for the transcriptional activation of a complex genomic locus may be identified by any of a number of methods known in the art. For instance, the skilled addressee may use bioinformatics methods to identify the sequence of a regulatory element (e.g. a promoter) for transcriptional activation of a complex genomic locus using a computer software, such as GENCODE or the like. The skilled person may identify promoter sequences proximal and distal to each gene of a complex locus using information on sequence conservation and transcriptional start site (TSS) data (e.g. FANTOM CAGE-Seq data). sgRNA may be identified in the genomic region −2 kbp, −1 kbp, −500 bp. −300 bp, −250 bp, −200 bp, −150 bp or −100 bp to 0 bp from each identified TSS in each locus using online available tools (e.g. CHOPCHOP). In a preferred embodiment, the sgRNA may be identified in the genomic region −300 bp to 0 bp from each identified TSS in each locus. In addition, the construct for use in the method as disclosed herein and the vector as disclosed herein enable manipulation of gene expression in cells with low transfectability, such as primary cells (e.g. primary SMCs), which is typically difficult to achieve with existing methods. Surprisingly, by using two exemplar complex genomic loci comprising multiple promoters and noncoding genes, the inventors have identified that activating a promoter upstream of the TSS of the genes can transcribe multiple transcripts derived from the complex genomic locus. As the expression of a complex genomic locus, such as lncRNAs and co-localised microRNAs, are typically regulated by multiple promoters within the same locus, it was unexpected that targeting a single promoter would result in activation of an entire complex genomic locus.

Single guide RNA as known in the art refers to a specific RNA sequence that recognises the target region of interest, such as a promoter of a complex genomic locus, which directs the Cas nuclease to the genomic locus of interest. The guide RNA typically comprises two components, a crispr RNA (crRNA) and a trans-activating crRNA (tracrRNA). The crRNA typically comprises 17-20 nucleotides and is complementary to the target DNA. The tracrRNA serves as a binding scaffold for the Cas nuclease.

The target or target sequence refers to a sequence to which a guide sequence is designed to have complementarity, where hybridisation between a target sequence and a guide sequence promotes the formation of a CRISPR complex. Full complementarity is not necessarily required, provided there is sufficient complementarity to cause hybridisation and promote formation of a CRISPR complex. A target sequence may comprise any polynucleotide, such as DNA or RNA polynucleotides.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogues thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogues. If present, modifications to the nucleotide structure may be imparted before or after assembly of the polymer. The sequence of nucleotides may be interrupted by non-nucleotide components. A polynucleotide may be further modified after polymerization, such as by conjugation with a labelling component.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridise under stringent conditions.

“Hybridisation” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridising strand, or any combination of these. A hybridisation reaction may constitute a step in a more extensive process, such as the initiation of PCR, or the cleavage of a polynucleotide by an enzyme. A sequence capable of hybridising with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this disclosure, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a complex genomic locus” or “gene expression” is the process by which information from a gene is used in the synthesis of a functional gene product. The products of gene expression are often mRNA encoding proteins, but in non-protein coding genes such as miRNA, lncRNA, rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

In a preferred embodiment, the deactivated Cas, such as Cas9 (dCas9), as disclosed herein may be fused to one or more transcriptional activators for the activation of the target complex genomic locus. In one embodiment, the deactivated Cas is fused to a tripartite fusion of three transcription activation domains VP64, p65 and Rta. In one embodiment, dCas9 is fused to a tripartite fusion of three transcription activation domains VP64, p65 and Rta. In some embodiments, the deactivated Cas may be fused to a scaffold that recruits one or more activator peptides or proteins.

Previously, the inventors have demonstrated that the expression of these lncRNA-microRNA loci is downregulated during the pathophysiology of vascular remodelling in atherosclerosis and pulmonary arterial hypertension (PAH). Valuable examples implicated in such pathophysiology are two multi-transcript lncRNA-microRNA loci, CARMN/miR-143/145 (19 isoforms) and MIR503HG/miR-424/miR-503 (5 isoforms). The expression of both lncRNA and microRNA are likely to be important for the maintenance of smooth muscle cell (SMC) and endothelial cell (EC) identity. By upregulating the expression levels of the lncRNA and microRNAs simultaneously, it is envisaged that it may be possible to maintain physiological cellular function and prevent pathological remodelling in disease.

The lncRNA CARMN has 24 transcriptional isoforms including the pri-microRNA transcripts encoding for miR-143 and miR-145. The expression of CARMN, miR-143 and miR-145 is necessary for the maintenance of SMC phenotype under normal physiological conditions, while their downregulation leads to loss of SMC identity. SMC identity is typically lost during the advancement of vascular remodelling towards a pro-pathological process. Therefore, to block or prevent its progression, it is envisaged that a global intervention is likely to be required to restore the expression of the entire locus to at least a therapeutically effective level. While using other approaches this could not be achieved, the relevance of the present strategy is represented by the contemporaneous overexpression of CARMN transcripts, miR-143 and miR-145 by simply targeting the promoter. This requires only one intervention and, contrarily to the other methods, it can be achieved with one vector. Moreover, the use of vectors, such as adenoviral vectors, for the delivery of CRISPR components allows the application of this strategy to multiple cellular contexts and various therapeutic applications.

In one embodiment, the construct for use in the method as disclosed herein is used to target complex genomic loci associated with cardiovascular disease. In one embodiment, the construct of the present disclosure may be used to target a complex genomic locus comprising the lncRNA H19 and miR-675¹¹, the lncRNA LEADeR host gene for miR-205¹², the host gene MIR100HG and miR-100/miR-let7a2/miR-125b1 embedded in the same locus¹³, or the protein coding gene Myosin encoding for co-located intronic microRNAs miR-208b, miR-499¹⁴, for example. In one embodiment, the construct for use in the method as disclosed herein is used to target the complex genomic loci comprising CARMN/miR-143/miR-145 or MIR503HG/miR-424/miR-503. In a particular embodiment, the construct for use in the method according to the present disclosure for targeting CARMN/miR-143/miR-145 or MIR503HG/miR-424/miR-503 comprises a sgRNA selected from SEQ ID NO: 31 to SEQ ID NO: 34.

In one embodiment, the construct for use in the method of the present disclosure is a component of a vector. Vector refers to a nucleic acid molecule capable of transporting another nucleic acid, such as the construct as disclosed herein, to which it is linked. Vectors include, but are not limited to, nucleic acid molecules that are single-stranded, double-stranded, or partially double-stranded; nucleic acid molecules that comprise one or more free ends, circular vector; nucleic acid molecules that comprise DNA, RNA or both; and other varieties of polynucleotides known in the art. In an alternative embodiment, the construct may comprise a component of a plasmid. Plasmid refers to a circular double-stranded DNA in which additional DNA segments can be inserted, such as by standard molecular cloning techniques.

Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Moreover, certain vectors are capable of directing the expression of genes to which they are operatively-linked. Such vectors, often in the form of plasmids can comprise a nucleic acid of the invention in a form suitable for expression of the nucleic acid in a host cell, which means that the vectors include one or more regulatory elements, which may be selected on the basis of the host cells to be used for expression, that is operatively-linked to the nucleic acid sequence to be expressed. “Operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In some embodiments, a vector comprises a regulatory element operably linked to an enzyme-coding sequence encoding a deactivated CRISPR enzyme, such as a deactivated Cas protein.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these.

In a preferred embodiment, the vector is a viral vector. Virally-derived DNA or RNA sequences are present in the viral vector for packaging into a virus. Viral vectors also include polynucleotides carried by a virus for transfection into a host cell. Certain vectors are capable of autonomous replication in a host cell into which they are introduced. Other vectors are integrated into the genome of a host cell upon introduction into the host cell, and thereby are replicated along with the host genome. Certain vectors are capable of directing the expression of genes to which they are operatively-linked. Viral vectors of the present disclosure may be selected from: an adenoviral vector, a lentiviral vector, a retroviral vector, an adeno-associated viral vector, a baculoviral vector, a vaccinia viral vector or a herpes simplex viral vector. In a preferred embodiment, the construct as disclosed herein is a component of an adenoviral vector. In another embodiment, the construct as disclosed herein is a component of a lentiviral vector.

The cell to which the construct or the vector comprising the construct is introduced is typically a eukaryotic cell. In one embodiment, the construct for use in the method as disclosed herein comprises a cell, which may be a eukaryotic cell. In one embodiment, the cell may be a mammalian cell. In one embodiment, the cell may be a vascular cell, such as an endothelial cell, a smooth muscle cell, or a fibroblast. In one embodiment, the cell may be a collection of cells, a tissue or an organ that comprises an endothelial cell, a smooth muscle cell, or a fibroblast or a combination thereof. The construct or vector comprising the construct may be introduced to the cell by various methods known in the art, such as transfection. Transfection methods known in the art include, but are not limited to, virus-mediated transfection, cationic polymer transfection, calcium phosphate transfection, cationic lipid transfection, electroporation, sonoporation, for example.

In one embodiment, the construct or vector for use in the method as described herein may be for use in an in vitro, in vivo or ex vivo method. In one embodiment, the construct or vector for use in the method as described herein is for use in an in vivo method. In one embodiment, the construct or vector for use in the method as described herein is for use in an ex vivo method. In one embodiment, the construct or vector for use in the method as described herein is for use in an in vitro method.

In another aspect, there is provided a vector for transcriptional activation of a complex genomic locus, wherein the vector comprises (i) a single guide RNA targeting a regulatory element of the said complex genomic locus, (ii) a deactivated Cas and (iii) one or more regulatory elements for the expression of the said single guide RNA and the said deactivated Cas.

In some embodiments, there is provided a vector for transcriptional activation of a complex genomic locus, wherein the vector comprises (i) a single guide RNA targeting a regulatory element of the said complex genomic locus, (ii) a first regulatory element for the expression of the said single guide RNA, (iii) a deactivated Cas and (iv) a second regulatory element for the expression of the said deactivated Cas.

In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Bil., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

In some embodiments, the first and the second regulatory element of the abovementioned vector may be selected from U6, EFS, CBh, H1, RSV, CMV or SV40 promoter. In a preferred embodiment, the first and the second regulatory elements may be selected from U6 and EFS. In an alternative embodiment, it is possible to use a cell type-specific promoter to restrict the expression of the sgRNA only in cells where the promoter is active. One example is represented by Myh11 promoter which is highly expressed in vascular smooth muscle cells. In this case, 2^{nd} or 3^{rd} generation adenovirus should be used as the length of the expression cassette exceeds the limit of AAV, lentivirus or 1^{st} generation adenoviruses. In one embodiment, the first and/or second regulatory element may be Myh11 promoter.

In one embodiment, the vector of the present disclosure encodes a deactivated Cas fused to one or more transcriptional activators, which typically comprises VP64, p65 and Rta (VPR). In some embodiments, the dCas9 is fused to the transcriptional activators VP64, p65 and Rta (VPR).

In one embodiment, the vector of the present disclosure comprises a single guide RNA which targets transcriptional activation of one or more noncoding gene(s), multi-transcript noncoding gene(s), or a combination thereof. In one embodiment, the noncoding gene(s) may comprise microRNA, lncRNA, pri-microRNA, siRNA, piRNA, snoRNA, snRNA, exRNA, scaRNA or a combination thereof. In certain embodiments, the noncoding gene(s) comprise long non-coding RNA, microRNA and/or pri-microRNA, or a combination thereof.

In some embodiments, the complex genomic locus for transcriptional activation may comprise CARMN/miR-143/miR-145 or MIR503HG/miR-424/miR-503. In some embodiments, a single guide RNA may be selected from SEQ ID NO: 31 to SEQ ID NO: 34 for the transcriptional activation of the complex genomic locus comprising CARMN/miR-143/miR-145 or MIR503HG/miR-424/miR-503.

In one embodiments, the vector may be a viral vector. The viral vector may be selected from an adenoviral vector, a lentiviral vector, a retroviral vector, an adeno-associated viral vector, a baculoviral vector, a vaccinia viral vector or a herpes simplex viral vector. In a preferred embodiment, the vector comprises an adenoviral vector. In an alternative embodiment, the vector comprises a lentiviral vector.

In one embodiment, there is provided a construct or vector for transcriptional activation of a complex genomic locus as disclosed herein for use in the treatment of disease. In some embodiments, the construct or vector may be provided for use as a medicament. In one embodiment, the vector for transcriptional activation of a complex genomic locus as disclosed herein may be for use in the treatment of a vascular disease. In a preferred embodiment, the vector for transcriptional activation of a complex genomic locus as disclosed herein may be for use in the treatment of atherosclerosis and/or pulmonary arterial hypertension.

In one embodiment, the construct for use in the method of transcriptional activation of a complex genomic locus or the vector as disclosed herein, may be for use in the prevention and/or regulation of pathological vascular remodelling, wherein the vector is introduced to a vascular cell.

In one embodiment, the vascular cell may be an endothelial cell, a smooth muscle cell or a fibroblast. In some instances, the construct or vector may be introduced to a collection of cells, a tissue or an organ comprising an endothelial cell, a smooth muscle cell, a fibroblast or a combination thereof.

In another aspect, there is provided a cell comprising transcriptional activation of a complex genomic locus using the construct for use in the method as described herein, wherein the cell is transfected with a vector as described herein. In one embodiment, the cell is a mammalian cell. In a preferred embodiment, the cell is a vascular cell. The vascular cell may comprise an endothelial cell, a smooth muscle cell, a fibroblast or a combination thereof.

In one embodiment, the construct or vector of the present disclosure may be provided for use in ex vivo gene therapy. In some embodiments, the construct or vector of the present disclosure may be used to transcriptionally activate a complex genomic locus in a cell ex vivo for transplantation. In one embodiment, the cell may be a vascular cell, such as an endothelial cell, a smooth muscle cell, or a fibroblast. In one embodiment, the cell may be a collection of cells, a tissue or an organ that comprises an endothelial cell, a smooth muscle cell, or a fibroblast or a combination thereof. In an alternative embodiment, the construct or vector may be used in an in vivo method for use in the treatment of a vascular disease, such as atherosclerosis and/or pulmonary arterial hypertension.

A subject to be administered the construct or vector as described herein may include any human or animal subject with a vascular disease. In some instances, the subject may be any human or animal predisposed and/or susceptible to developing a vascular disease, such as atherosclerosis and/or pulmonary arterial hypertension, wherein the vascular disease may be treated, ameliorated, or prevented with the use of the construct or vector as disclosed herein.

In one teaching, the vector as described herein may be provided as a pharmaceutical composition, formulated with at least one pharmaceutically acceptable excipient thereof. In one embodiment, an acceptable excipient may be selected from water, saline (e.g. phosphate-buffered saline), human serum albumin, dextrose, trehalose, sucrose, mannitol, sorbitol, polysorbate 20, polysorbate 80, glycerol, ethanol, polyethylene glycol, or the like and combinations thereof. In some embodiments, the pharmaceutical composition may comprise one or more excipients that promote cellular uptake of the vector.

The pharmaceutical composition may comprise a therapeutically or prophylactically effective amount of one or more vectors as disclosed herein. In one embodiment, the pharmaceutical composition may comprise an effective amount of any one or more vectors of the present disclosure, or a combination thereof. A therapeutically or prophylactically effective amount of one or more vectors refers to an amount or concentration of a vector that is sufficient to transcriptionally activate a complex genomic locus, and thereby restore physiological cellular function and/or prevent pathological cellular state(s).

In one embodiment, the pharmaceutical composition may optionally further comprise one or more pharmaceutically acceptable stabilisers, wetting agents, emulsifiers, salts, buffers and/or adjuvants known in the art.

The vector may be formulated into the composition as non-ionic or salt forms. Pharmaceutically acceptable salt refers to a salt of a compound that is pharmaceutically acceptable and that possesses, or can be converted to a form that possesses, the desired pharmacological activity of the parent compound. Such salts include acid addition salts formed with inorganic acids, such as hydrochloric acid, sulphuric acid, nitric acid, phosphoric acid and the like; or formed with organic acids, such as acetic acid, citric acid, glucoheptonic acid, lactic acid, for example.

In one teaching, the vector as disclosed herein may be provided in the form of a kit comprising one or more vectors for transcriptional activation of one or more complex genomic loci. For instance, the kit may comprise one or more vectors for transcriptional activation of one or more complex genomic loci for use in in vitro assays. In one embodiment, the kit may comprise one or more vectors for transcriptional activation of one or more complex genomic loci for use in ex vivo applications. In some embodiments, the kit may comprise one or more vectors for transcriptional activation of one or more complex genomic loci in vivo.

### DETAILED DESCRIPTION

The present disclosure is further described by way of example and with reference to the figures, which show:

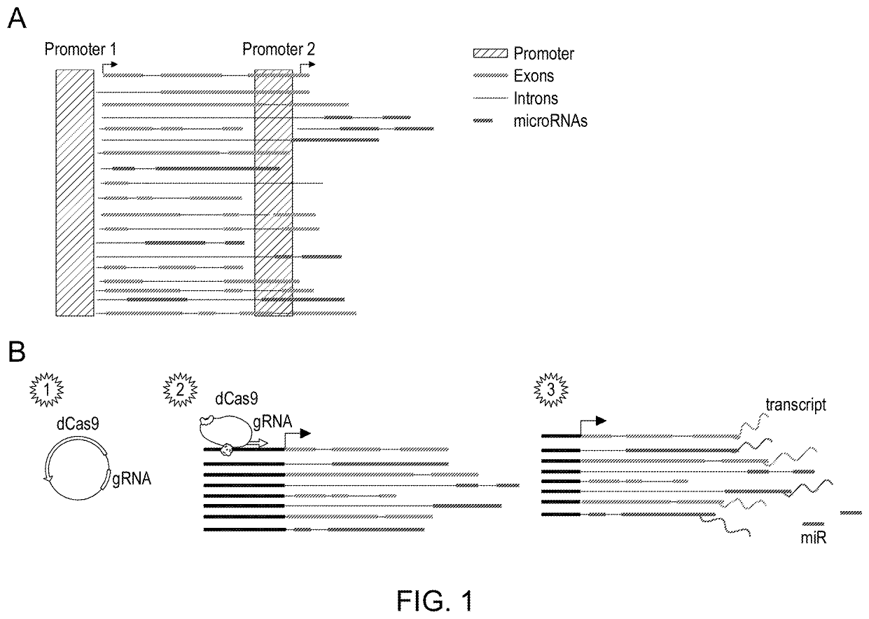

FIG. 1. Schematic illustration of the strategy to simultaneously activate host gene loci. A) Example of a complex multi-transcript locus including different primary transcripts (T1, T2) with different transcriptional start sites and different promoters. The locus also includes the primary transcript encoding for microRNAs. B) Phases required to obtain transcriptional activation of complex loci. “Phase1”: production of a unique vector expressing the dCas9 (mutated endonuclease) and single guide RNA (sgRNA). In particular, a unique sgRNA is designed to target the main promoter upstream the transcripts belonging to the same locus. “Phase 2”: the binding of the dCas9/sgRNA complex to the promoter will favor the transcriptional activation of the desired locus. “Phase 3”: the whole panel of transcripts produced by the same promoter (long noncoding RNAs, microRNAs) can be simultaneously activated.

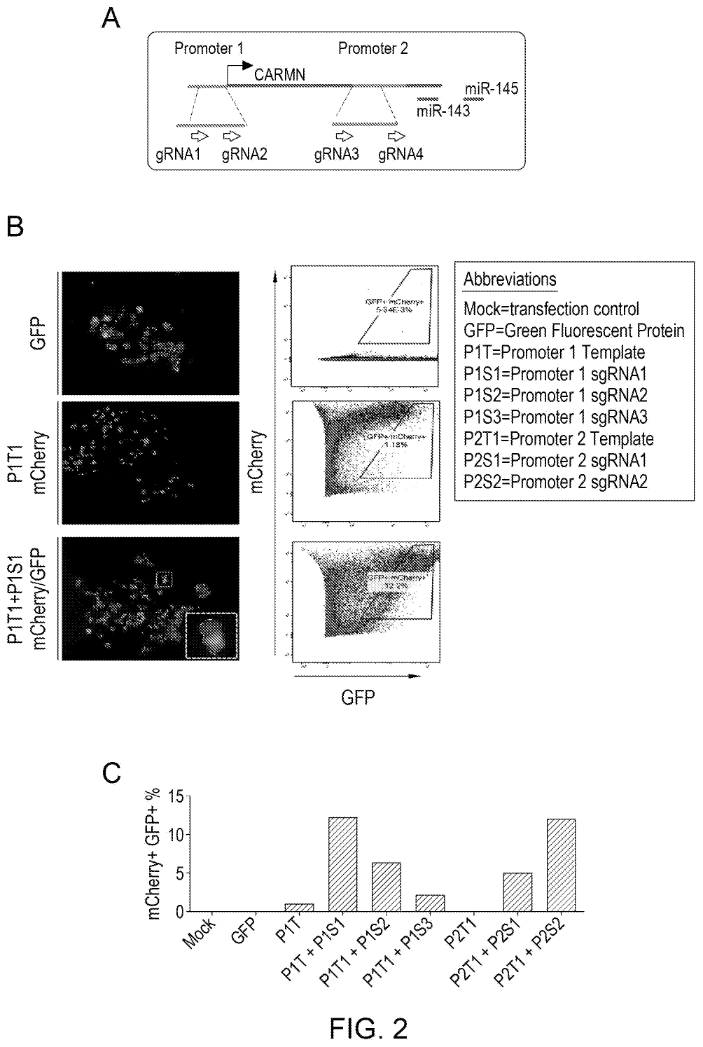

FIG. 2. Validation of sgRNA targeting CARMN promoters. A) Simplified representation of sgRNAs targeting CARMN locus, which includes the lncRNA CARMN (24 transcriptional isoforms), microRNA-143 and microRNA-145 (GENCODE v39). Multiple sgRNAs were designed targeting CARMN promoter 1 (P1) or promoter 2 (P2). To test the efficiency of binding of sgRNAs, HEK293 cells were transfected with 2 plasmids expressing Cas9/sgRNA, in-frame mCherry and out-of-frame GFP. The selective targeting of the Cas9/sgRNA to the promoter, favored the switch in-frame of GFP protein. B) Micrographs and FACS plots of double positive cells at 48 hours post transfection. C) Percentages of mCherry/GFP positive cells indicating the efficiency of sgRNAs obtained using Flow Cytometry. One sgRNA targeting each promoter was selected (P1S1 and P2S4).

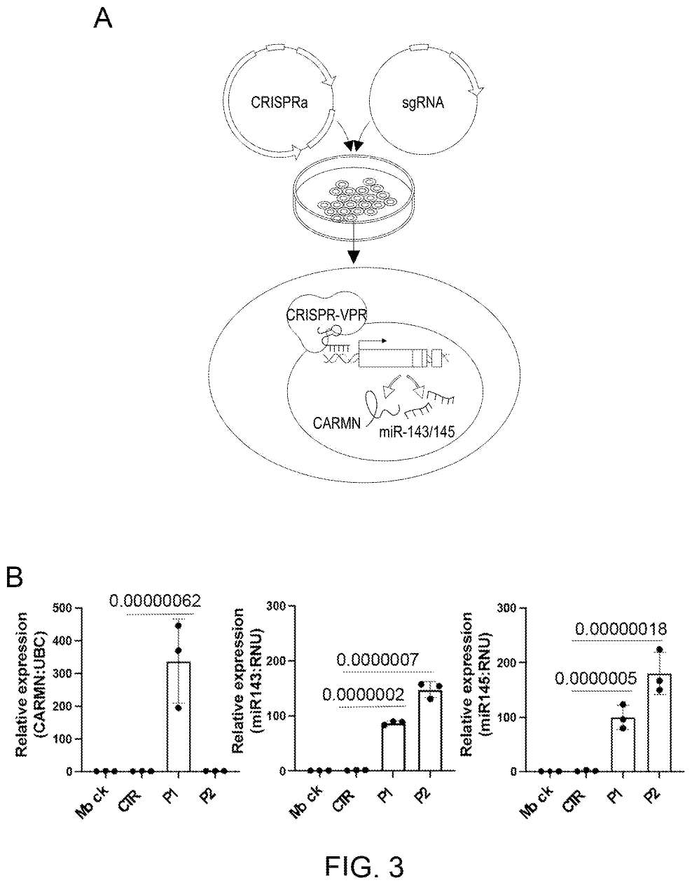

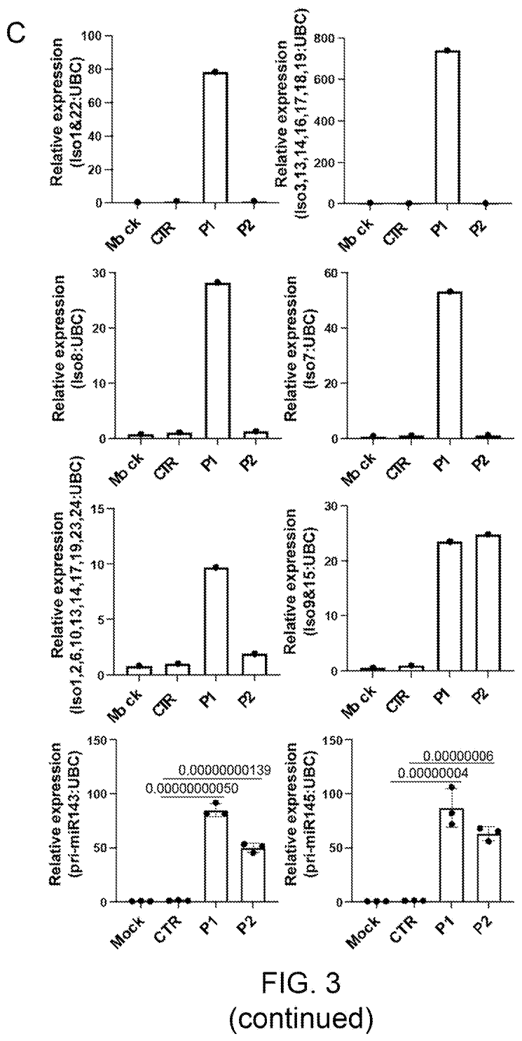

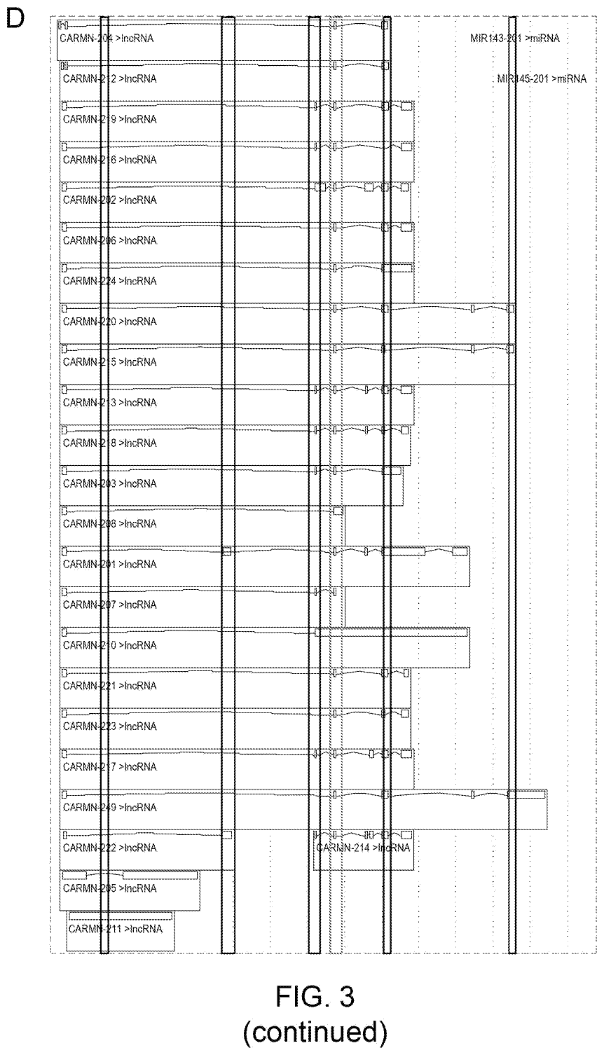

FIG. 3. CRISPRa-mediated targeting of CARMN promoter activates the whole locus. A) Strategy used to activate CARMN/miR-143/145 using CRISPR activator (CRISPRa) in HEK293 cells. B) Expression data of CARMN (using common primers), miR-143 and miR-145 (n=3 biological). C) Expression of CARMN transcripts (n=1 biological) and pri-microRNAs (n=3 biological). D) Representation of CARMN locus (GENCODE v39), including CARMN (24 transcripts) and miRNAs. The boxes indicate the regions amplified by primers used in C and the exon amplified by common primers used in B. UBC and RNU48 were used as housekeeper genes for qRT-PCR analysis. Statistical analysis and exact p-values were obtained using One-way Anova.

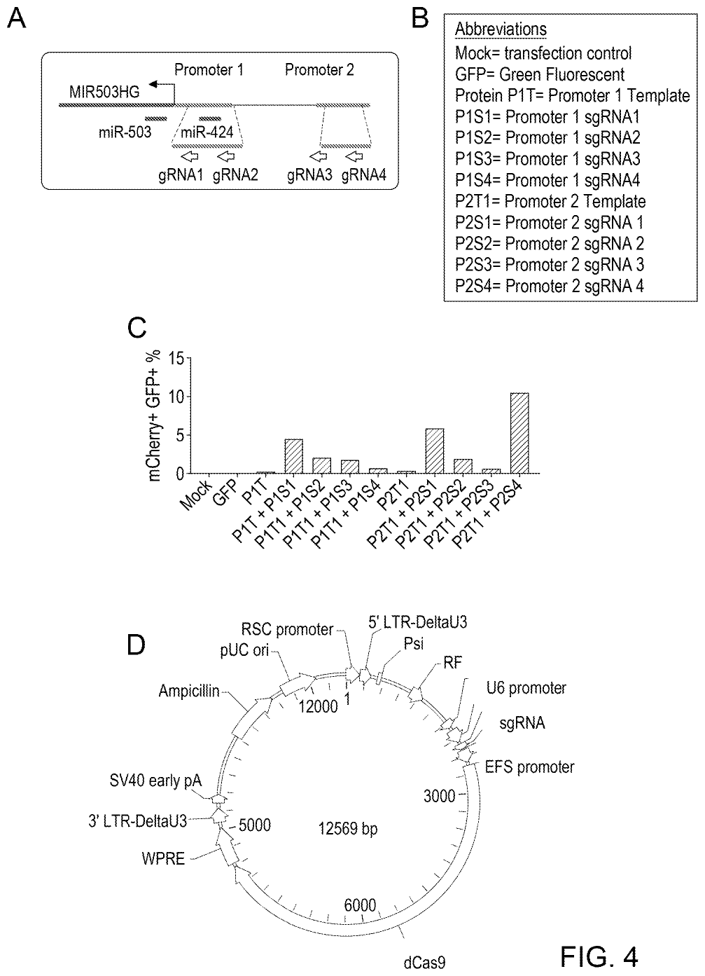

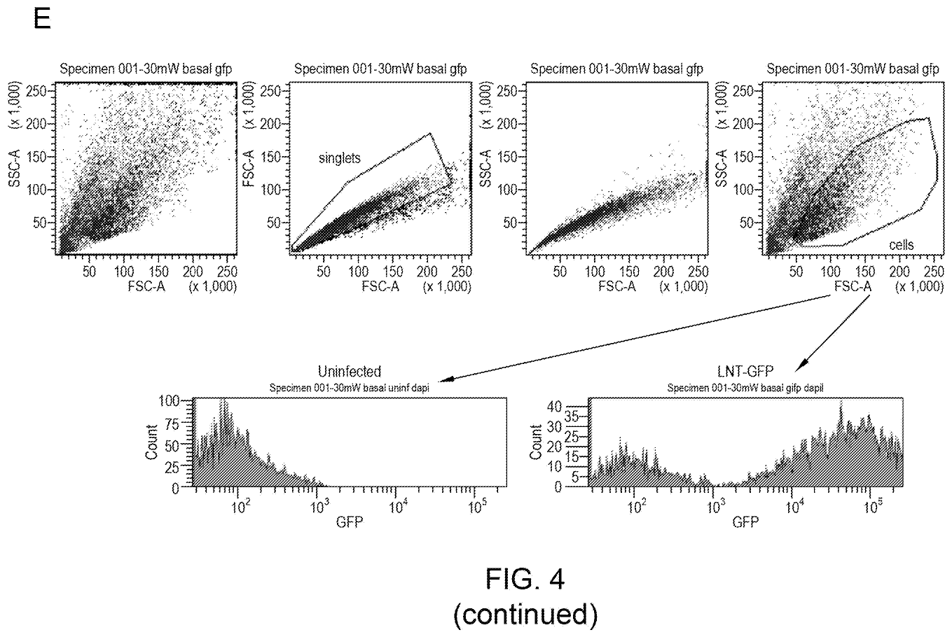

FIG. 4. Validation of sgRNAs activating MIR503HG locus in HEK293 cells. A) Simplified representation of MIR503HG locus (GENCODE v39), which includes the lncRNA MIR503HG (5 transcripts), microRNA miR-424 and miR-503. The locus includes 2 promoter regions, one distal upstream MIR503HG (promoter 2), and one proximal to the transcriptional start site of MIR503HG (promoter 1). Multiple sgRNAs were designed to target both regions and cloned into the same system used in FIG. 2. Single sgRNA 1 (P1S1) and 4 (P2S4) were selected for lentiviral expression. B) List of abbreviations used in C. C) Percentage of mCherry/GFP positive cells at 48 hours post transfection with Cas9/sgRNA machinery. D) Lentiviral map including the components required for CRISPRa-mediated activation of MIR503HG. E) FACS plots of primary endothelial cells (HUVEC) infected with a lentivirus expressing GFP protein (LNT-GFP) versus uninfected cells.

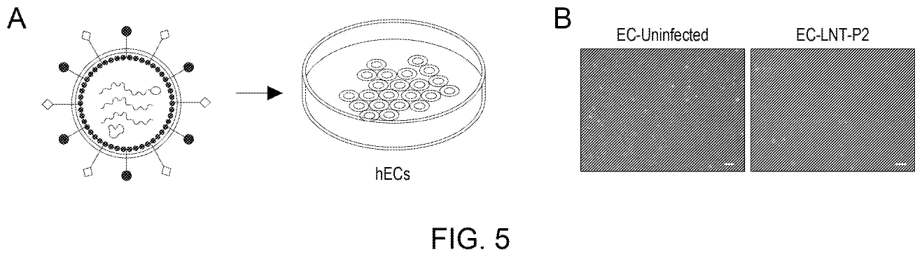

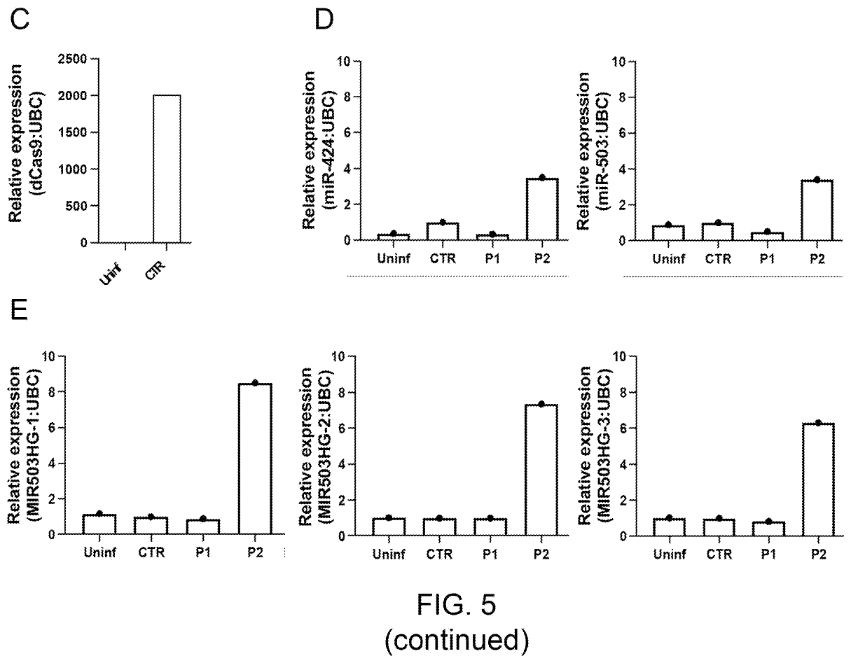

FIG. 5. Lentiviral-mediated activation of MIR503HG in primary endothelial cells. A) Cartoon illustrating the lentiviral virus (LNT) including the dCas9/sgRNA construct targeting MIR503HG delivered to human primary endothelial cells (HUVEC). B) Micrographs of HUVEC at 48 hours post infection with lentivirus expressing sgRNA targeting promoter 2 (LNT-P2) and uninfected cells (scale bar 100 um). C) Expression levels of dCas9 transcript at 48 hours post infection from lentiviral infection versus uninfected cells (n=1 biological). D) Graph indicating qRT-PCR results of miR-424 and miR-503 at 48 hours post infection with a lentiviral vector expressing sgRNA targeting promoter 1 (P1) or promoter2 (P2) versus control lentivirus (CTR) or uninfected cells (n=1 biological). E) Expression levels of MIR503HG transcripts at 48 hours post infection with LNT-P1, LNT-P2, LNT-CTR or uninfected cells (n=1 biological). UBC and RNU48 were used as housekeeping genes for qRT-PCR analysis.

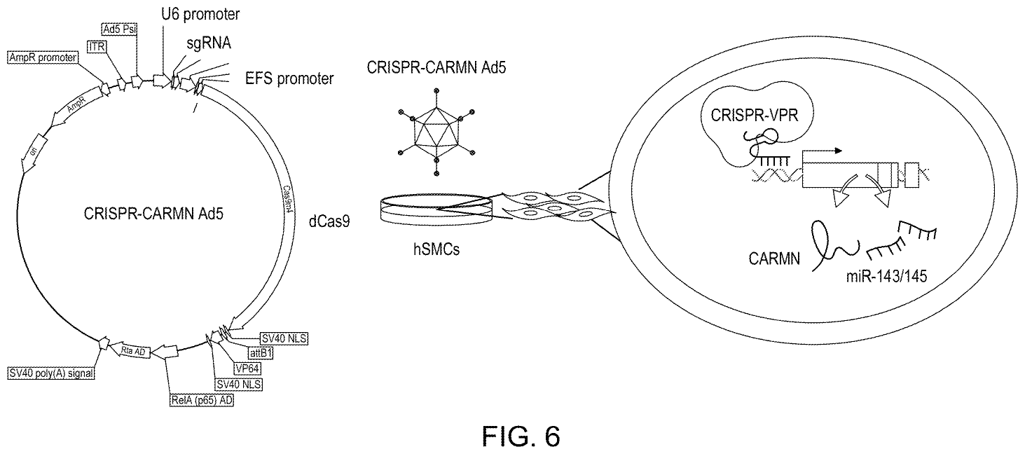

FIG. 6. Adenovirus 5 map for CRISPRa-mediated activation of CARMN/miR-143/145 in human primary cells. A) Schematic representation of adenovirus serotype 5 (Ad5) expressing the components required to activate CARMN and microRNAs in primary SMCs. This vector expresses the sgRNA targeting promoter 1 for the simultaneous activation of CARMN and microRNAs. B) Cartoon representing the use of Adenoviral vector serotype 5 transducing primary cells in vitro, to simultaneously activate multiple isoforms of CARMN, miR-143 and miR-145.

### METHODS

Design of Single Guide RNA (sgRNA)

CARMN and MIR503HG promoter sequences (proximal and distal to each locus) were identified using information on sequence conservation and Transcriptional Start Site (TSS) data (FANTOM CAGE-Seq data). Multiple sgRNAs were designed in the genomic region −300 to 0 bp from each identified TSS in each locus using online available tools (CHOPCHOP). The sequences of selected sgRNA recognising CARMN and MIR503HG promoters are listed in Table III.

Cloning and Amplification of Vector Constructs into a CRISPR/Cas9 System

At least 3 sgRNAs targeting the promoter of each locus (CARMN and MIR503HG) were selected to be tested for specificity of binding using CRISPR/Cas9 system. Forward and reverse sgRNA oligonucleotides (IDT) were designed with overhangs compatible for cloning into BbsI site in pX330 plasmid (Addgene, 110403). Following resuspension to 100 uM in IDT Duplex Buffer (IDT #11-05-01-03), forward and reverse oligonucleotides were annealed by incubation for 5 min at 95° C. and phosphorylated using the T4 polynucleotide kinase (ThermoFisher Scientific, EK0032) by incubating them for 30 minutes at 37° C. The same procedure was performed for the promoter templates oligonucleotides: forward and reverse oligonucleotides were designed with overhangs to be cloned into Esp3l site in pBS SK mCherry-EGFP (Addgene, 54322). The ligation of annealed dsgRNA oligonucleotides into pX330 or pBS SK mCherry-EGFP vector was performed using 5 ng of digested vector, 10×DNA ligase buffer (NEB #B0202A) and T4 ligase (NEB #M0202T). The ligation reaction was performed for 1 h at room temperature. Following ligation, 5 μl from each ligated vector (containing sgRNA or promoter template) was used to transform DH5-alpha competent cells (NEB, C2987H) for 30 minutes on ice. Heat shock was induced by heating cells at 42° C. for 45 sec followed by incubation on ice for 2 min. Cells were then incubated with SOC medium (NEB B9020S) at 37° C. at 220 rpm for 1.5 h, and then spined down at 5000 rpm for 10 min at room temperature and plated on ampicillin plates overnight. The following day, 4 colonies from each construct were inoculated in 5 ml of LB medium supplemented with antibiotics and cells were left to grow overnight at 37° C. with shaking. Isolation of bacterial DNA was performed using DNA Miniprep kit (Qiagen kit, 27106) following manufacturer's instructions. Sequencing of the inserts was performed using lug of each plasmid vector and human forward U6 primer (Source Bioscience).

Cloning and Amplification of Vector Constructs into a CRISPRa System

The guide RNA selectively targeting the promoter of each locus (CARMN and MIR503HG) was selected to be used for following activation experiments with CRISPR activator system. Forward and reverse sgRNA oligonucleotides (IDT) were designed with overhangs compatible for cloning into BbsI site and BsmBI-v2 of the B52 plasmid (Addgene, 100708). Following resuspension to 100 uM in IDT Duplex Buffer (IDT #11-05-01-03), forward and reverse oligonucleotides were annealed by incubation for 5 min at 95° C. and phosphorylated using the T4 polynucleotide kinase (ThermoFisher Scientific, EK0032) by incubating them for 30 minutes at 37° C. For the ligation of the phosphorylated dsgRNA oligonucleotides into the B52 vector, 50 ng of digested vector and 37 ng of dsgRNA were ligated by incubation at 22° C. for 5 min using the Quick Ligation™ kit (Neb, M2200L). Following ligation, 2 μl from each ligated vector (containing dsgRNA or promoter template) were used to transform DH5-alpha competent cells (NEB, C2987H) for 30 minutes on ice. Heat shock was induced by heating cells at 42° C. for 45 sec followed by incubation on ice for 2 min. Cells were then incubated with SOC medium (NEB B9020S) at 37° C. t 220 rpm for 1.5 h, and then spined down at 5000 rpm for 10 min at room temperature and plated on ampicillin plates overnight. The following day, 4 colonies from each construct were inoculated in 5 ml of LB medium supplemented with antibiotics and cells were left to grow overnight at 37° C. with shaking. Isolation of bacterial DNA was performed using DNA Miniprep kit (Qiagen kit, 27106) following manufacturer's instructions. Sequencing of the inserts was performed using lug of each plasmid vector and human forward U6 primer (Source Bioscience).

### Human Cell Culture

Human Embryonic Kidney 293 (HEK293) were cultured in DMEM medium (Gibco #11965092) supplemented with 10% foetal bovine serum (Life Technologies, Paisley, UK), 50 μg/mL penicillin and 50 μg/mL streptomycin (Gibco, Paisley, UK). Cells were maintained in culture in complete medium in humidified atmosphere 37° C. (5% CO2) and passaged when reached 95% confluence. Human Umbilical Vein Endothelial Cells (HUVEC #C2519A) cells were purchased from Lonza (Basel, Switzerland) and maintained in endothelial cell growth medium (EGM-2 BulletKit™) (Lonza, Basel, Switzerland) supplemented with foetal bovine serum (FBS) (10%, Life Technologies, Paisley, UK) and 50 μg/mL Penicillin-Streptomycin (P/S) (100 U/ml) (Gibco, Paisley, UK). Cells were maintained in culture in complete medium in humidified atmosphere 37° C. (5% C02) and used until passage 6.

### Transfection of HEK293T Cells

HEK293T cells were plated at a confluence of 2×10⁵ cells/well into 6-well plate in complete medium: DMEM (Gibco #11965092), 10% FBS (10%, Life Technologies, Paisley, UK) and 50 μg/mL Penicillin-Streptomycin (P/S) (100 U/ml) (Gibco, Paisley, UK). The following day cells were transfected using 1 ml of OptiMem medium (Gibco, 31985070) and 3 μl of Lipofectamine 2000 (ThermoFisher Scientific, 11668019) as transfection reagent per well. In particular, lug of each plasmid for the co-transfection of sgRNA-pX330 plasmid with promoter template-pBS plasmid, or lug and 200 ng in the case of co-transfection of dCas9-VPR plasmid (Addgene, 63798) with dsgRNA plasmid (Addgene, 100708) were co-transfected into each well. Following 6 h from the transfection, 2 ml of fresh complete medium was added to cells. The following day, medium was replaced with fresh complete medium. At 48 h from the transfection, cells were harvested for following downstream analysis.

RNA Extraction from Cultured Cells

Total RNA was extracted using miRNeasy kit (Qiagen, Hilden, Germany Cat: 217004) following manufacturer's instructions. After treatment, cells were washed once in PBS and harvested in 700 μl of Qiazol lysis reagent (Qiagen, Cat:79306). In the case of tissues, fresh tissues were first homogenised in Qiazol reagent and processed using a tissue homogeniser. Chloroform was added to each sample (140 ul) and after 3 min of incubation, samples were centrifuged for 20 min at 12.000×g at 4° C. The supernatant phase was then collected into a new 1.5 μl tube and 550 μl of 100% ethanol was then added to each sample. Samples were then placed into cartilage columns provided with the kit and centrifuged at 8.000×g for 1 min. The flow through solution was discarded and 350 μl of RWT buffer (previously combined with ethanol as manufacturer's instructions) was added. After centrifugation (at 8.000×g for 1 min), samples were treated for 10 min with DNase enzyme (RNase-free DNase set, Qiagen Cat:79256) as indicated in the manufacturer's instructions at room temperature (80 μl of a mix of DNase enzyme and RDD buffer was added to each sample). Samples were then washed again with 350 μl of RWT. After centrifugation, the eluted buffer was discarded and 2×500 μl of RPE buffer (previously combined with ethanol) were added to each sample. Columns were then replaced with new 2 ml tubes provided with the kit for a step of centrifugation at 8.000×g for 1 min to allow any residual RPE buffer to be discarded. RNA was then eluded in 30 μl of RNase-free H2O and concentration was quantified by using Nanodrop 1000 spectrometer (Thermo Scientific, Parsley, UK) and stored into −80° C. for following analysis.

Gene Expression Analysis by qRT-PCR

CDNA for mRNA analysis of gene expression was synthesized from total RNA using the Multiscribe Reverse Transcriptase (Life technologies, Paisley, UK). CDNA for miRNA analysis was obtained from total RNA using specific reverse transcription primers according to the TaqMan MiRNA Assay protocol (Applied Biosystem, Foster City, CA, USA). Quantitative qRT-PCR was performed using Power SYBR green (Life technologies) with custom PCR primers (Eurofins Scientific, Ebersberg, Germany). Forward and reverse primer sequences for CARMN and MIR503HG are listed in table I. In the case of Sybr Green qRT-PCR, samples were subjected to 2 minutes at 50° C., 10 minutes at 95° C., 40 cycles of denaturation for 15 seconds at 95° C., 1 min at 60° C. In the case of TaqMan reaction performed with TaqMan probes (probe ID listed in Table II), qRT-PCR plate underwent to a first step of 2 min at 50° C. followed by 10 min at 95° C. and 40 cycles at 95° C. for 15 seconds to finish with 1 min at 60° C. Ubiquitin C (UBC) for gene expression and RNU48 for microRNA were selected as housekeeping genes because of their stability across all studied groups. Fold changes were calculated using the 2^{-ΔΔct} method.

### Flow Cytometry (FACS)

Cells were harvested 48 h post infection with lentiviral particles expressing GFP fluorescent protein or uninfected control cells. At 48 h from transfection medium was removed, cells were washed twice with sterile PBS, 300 μl of 1% trypsin (Gibco) were added into each well and cells were incubated at 37° C. for 2-3 minutes. At the end of the incubation, 700 μl of complete medium was added to neutralise the trypsin. Cells from each well were then centrifuged (1200×g, 5 minutes), medium was removed, and cells were resuspended in 500 μl of FACS buffer (PBS w/ 1% BSA & 1 mM EDTA). Cells in suspension were acquired using FACSCanto II and FACSdiva software (BD Bioscience), and FACS data were analyzed using Flow Jo software.

Primary Cell Infection with Lentiviral Particles

Primary endothelial cells (HUVEC) were seeded at a density of 3×10⁴ per well in a 12-well plate format in complete medium (EGM-2 BulletKit™ Lonza, Basel, Switzerland) supplemented with foetal bovine serum (FBS) (10%, Life Technologies, Paisley, UK) and 50 μg/mL Penicillin-Streptomycin (P/S) (100 U/ml) (Gibco, Paisley, UK). The following day, cells were infected at a Multiplicity of Infection of (MOI) 500, in complete medium (500 μl per well) using 5 ug/ml of Polybrene (Sigma-Aldrich, TR-1003-G) to enhance the efficiency of cell infection. Cells were incubated for 48 h in humidified atmosphere 37° C. (5% CO2), and then harvested for downstream analysis.

### Statistical Analysis of Experimental Data

Graphs are presented as bar charts of mean±standard error of the mean (SEM) with individual data points superimposed to show full data distribution. QRT-PCR data in graphs is shown as relative expression to housekeeping control as described by Livak and Schmittgen³. Statistical tests used to assess statistical significance is indicated in each figure legend with the precise p-value provided in the graphs where statistical significance was observed. All biological replicates correspond to independent experiments from distinct expansions and passage numbers, with technical replicates (precise replicate number indicated in the figure legends). As each experimental data set is an average of a large number of cultured cells, we assumed the data was normally distributed based on the central limit theorem. Statistical analysis of biological replicates was performed using one-way ANOVA with Bonferroni correction for multiple comparisons (>2 groups comparison). Statistical analysis was performed using GraphPad Prism 8.0.0.

TABLE I
Name and sequences of forward and reverse Sybr Green primers
amplifying CARMN and MIR503HG transcripts.

| Sybr Green primer | | SEQ |
|---|---|---|
| name | SEQUENCE | ID NO |
| CARMN_001_Fw | 5′ TGGGAATGGAAACCCTTGGT 3′ | 1 |
| CARMN_001_Rev | 5′ AAGCTGCATGTTCAAGGTCG 3′ | 2 |
| CARMN_003_Fw | 5′ CTGTAACAAGTGCACAGGCAA 3′ | 3 |
| CARMN_003_Rev | 5′ TTCTGTTGGGAGGCTTGGAGA 3′ | 4 |
| CARMN_005_FW | 5′ CCAGGAAAGGCAAGGGCGCTA 3′ | 5 |
| CARMN_005_Rev | 5′ TGGGAGCGGATGTGGGTGCCG 3′ | 6 |
| CARMN_006_Fw | 5′ GCTGTAACAAGTGCACAGGCA 3′ | 7 |
| CARMN_006_Rev | 5′ GTGGGCTCACAGTTCTGTCT 3′ | 8 |
| CARMN_007_Fw | 5′ GATCCAGAGTAGGAGGGAGCC 3′ | 9 |
| CARMN_007_Rev | 5′ GGCCCTTGAATCTGCTTGCC 3′ | 10 |
| CARMN_008_Fw | 5′ AGCCTGGAAGTGGCTGGATGT 3′ | 11 |
| CARMN_008_Rev | 5′ AAACGCATGCCTGATGGTGT 3′ | 12 |
| CARMN_009_Fw | 5′ GCTCCCAAAGCAGGAAGACC 3′ | 13 |
| CARMN_009_Rev | 5′ GCCCAACCTCACAAATCCTCT 3′ | 14 |
| CARMN_010_Fw | 5′ AATGCAGGAGGCATGGGCCA 3′ | 15 |
| CARMN_010_Rev | 5′ GCCACTTGAGTCAGTGATGGTG 3′ | 16 |
| CARMN _011_Fw | 5′ AAAAGTCAGAGGCTGTGGGAC 3′ | 17 |
| CARMN_011_Rew | 5′ TTGCCCACACAATGCCCTA 3′ | 18 |
| CARMN _012_Fw | 5′ TGCCTCTTCAGCTCATATAAG 3′ | 19 |
| CARMN _012_Rev | 5′ GCTAGCGCCCTTGCCTTTCCT 3′ | 20 |
| Common primer_Fw | 5′ CGCCATGCTGATGTCAGAGA 3′ | 21 |
| Common primer_Rev | 5′ GTTCTGTCTCCGGGCTGC 3′ | 22 |
| Ubc_Fw | 5′ TTGCCTTGACATTCTCGATG 3′ | 23 |
| Ubc_Rev | 5′ ATCGCTGTGATCGTCACTTG 3′ | 24 |
| 503HG_1_Fw | 5′ CCACAGGACAACGAAGAAAACC 3′ | 25 |
| 503HG_1_Rv | 5′ TACTCCTTTTCCAGTCCTCCCC 3′ | 26 |
| 503HG_2_Fw | 5′ GTGGAACCCCACACAGGAAA 3′ | 27 |
| 503HG_2_Rv | 5′ GGACAGTTGCCCATATTAACGG 3′ | 28 |
| 503HG_3_Fw | 5′ GGACCTGAGCTGTCGATTTCA 3′ | 29 |
| 503HG_3_Rv | 5′ GAGGGATGGAGGTGGCTTTA 3′ | 30 |

TABLE II
Name and sequences of TaqMan probes used to amplify the mature
sequence of microRNA (miR-143, miR-145, miR-424, miR-503) and
pri-microRNA transcripts (pri-miR-143 and pri-miR-145).

| TAQMAN PROBES | ASSAY ID |
|---|---|
| UBC | Hs01867132_s1 |
| Hsa-miR-143-3p | 477912_mir |
| Hsa-miR-145-5p | 477916_mir |
| RNU48 | ID 001006 |
| Hsa-miR-424-5p | ID: 000604 |
| Hsa-miR-503-5p | ID: 001048 |
| Hsa-miR-145 Pri-microRNA | Hs03303169_pri |
| Hsa-miR-143 Pri-microRNA | Hs03303166_pri |

TABLE III
Name and sequences of single guide RNA (sgRNA)
selectively binding CARMN and MIR503HG promoters.

| sgRNA name | sgRNA sequence | SEQ ID NO |
|---|---|---|
| CARMN_p1 | 5′ AAAGCAACATTCAGCACCGG 3′ | 31 |
| CARMN_p2 | 5′ GCTCCCAGCCCAGAGCAGCC 3′ | 32 |
| MIR503HG_p1 | 5′ ATGAAGGGGGATCGATCTGG 3′ | 33 |
| MIR503HG_p2 | 5′ TGTAGGGAAGGGGGTTGTGC 3′ | 34 |

### EXAMPLES

Design and Selection of sgRNAs Targeting CARMN and MIR503HG Promoters Using HEK293T Cells and CRISPR/Cas9 Technology

FIG. 1 represents the strategy adopted which is based on a novel CRISPR activator approach for the transcriptional activation of complex noncoding RNA loci in primary cells. An example of complex locus is represented in A where is shown a long noncoding RNA host gene for microRNAs. The complexity of this locus is given by the presence of multi-transcript lncRNA, the presence of microRNAs and of two promoter regions regulating the expression of these noncoding RNAs. The overexpression of genomic loci can be possible by ectopically providing the transcript of interest to the desired cells. However, this approach is not feasible in the case if complex genes, where it is required the simultaneous overexpression of all the components of the locus. In this approach, CRISPR activator technology (dCAS9-VPR), composed by inactive Cas9 (dCas9) and a single guide RNA (sgRNA) targeting the main promoter of the gene of interest, are adopted to develop a method for simultaneous transcriptional activation of different noncoding genes using a single vector. The main steps are represented in FIG. 1B, where the sgRNA designed to clone the promoter region is cloned into a plasmid together with the sCas9-VPR machinery. The transduction of cells with this unique vector induces the transcriptional activation of long noncoding RNA transcripts as well as microRNAs embedded in the same genomic locus.

FIG. 2 shows CRISPR/Cas9 strategy used to test sgRNAs for their specificity of binding to the promoter regions of CARMN locus. CARMN is a very complex lncRNA which includes 24 transcriptional isoforms and is host gene for miR-143/145. To simultaneously overexpress CARMN transcripts and microRNAs is not feasible with current methods. Therefore, we designed sgRNAs targeting the 2 promoter regions identified in this locus: promoter 1 (P1), proximal to CARMN transcriptional start site, and promoter 2 (P2), distal to CARMN and immediately upstream to miR-143 and miR-145 (A). We used a co-transfection strategy of HEK293T cells which we used as tool to easily manipulate gene expression via transfection of large transcripts. This would have not been possible in primary smooth muscle cells (SMC) due to their low transfectability. A plasmid expressing each single sgRNA in combination with Cas9 machinery was transfected in combination with a second plasmid expressing the promoter region recognized by the sgRNA, together with in-frame mCherry transcript and out-of-frame GFP protein transcript (B). The effective binding of the sgRNA to the promoter region of CARMN (promoter 1 or promoter 2) was able to switch in-frame the expression of GFP. AT 48-hours post transfection, cells were analyzed at Flow Cytometry and the percentage of double positive cells to mCherry and GFP were evaluated in each condition. One sgRNA per each promoter region was selected as the most efficient for following experiments.

### Assessment of CARMN/miR-143/145 Activation in Human Cell Line

After having selected the most efficient sgRNAs binding to CARMN promoters, we tested the effective transcriptional activation of them. To do so, we used the dCas9-VPR machinery which recruits transcriptional activators to the targeted promoter and induces transcriptional activation (FIG. 3A). Also in this case, we used HEK293T cells as tool to easily enhance CARMN locus expression. We observed that by targeting the main promoter, upstream CARMN and miRNAs, simultaneously activates the expression of the lncRNA CARMN, miRNA-143 and miRNA-145. Moreover, we have shown that the activation of promoter 2, downstream to CARMN, activates only miR-143/145 expression. A more detailed characterization of the activated transcripts following the targeting of both promoters, confirmed that the targeting of promoter 1 is enough to activate in concert the transcriptional isoforms as well as the pri-microRNAs (FIG. 3C, D).

Design and Production of Lentiviral Particles Expressing dCas9 and sgRNA in the Same Backbone

Having assessed the efficiency of our strategy, we applied it to another very complex noncoding RNA locus named MIR503HG (FIG. 4). This locus includes 5 transcripts belonging to the lncRNA MIR503HG which is host gene for 2 microRNAs, miR-424 and miR-503. Similarly to CARMN, MIR503HG locus includes 2 promoters, one upstream the whole locus (named promoter 2) and one just upstream the lncRNA, named promoter 1. We validated the sgRNAs targeting MIR503 promoters using the same strategy used for CARMN (FIG. 4A, B). After having identified the most effective sgRNAs we decided to include them into a unique lentiviral vector for the activation of MIR503HG in primary cells. The use of a gene delivery approach mediated by viral vectors overcomes the difficulty of transfecting primary cells with large plasmids. Therefore, we designed a novel lentiviral vector (FIG. 4D) which includes the dCas9-VPR and the sgRNA targeting the desired promoter region. First we tested the efficiency of transduction of this lentiviral vector by infecting primary endothelial cells. We have observed >70% positive cells to Green Fluorescent protein expressed by the lentiviral construct (FIG. 4E) versus uninfected cells.

### Assessment of MIR503HG Activation in Human Primary Cells

We then evaluated the efficiency of activation of MIR503HG locus by infecting primary ECs with a lentivirus targeting promoter 1 or promoter 2 (FIG. 5A). We did not observe significant cell toxicity following infection versus untreated cells (FIG. 5B). Moreover, we observed increased dCas9 transcript levels in infected versus control cells (FIG. 5C). Following the initial test of lentiviral transduction and expression efficiency, we assessed the activation of MIR503HG locus. QRT-PCR analysis shown transcriptional activation of MIR503HG, miR-424 and miR-503 following infection with sgRNA targeting promoter 2 versus control cells. While, the targeting of promoter 1 (upstream MIR503HG but downstream miRNAs) did not show activation, therefore confirming that simply targeting the upstream promoter is enough to activate the whole MIR503HG locus (FIG. 5D, E).

### Production of Adenoviral Particles for the Transcriptional Activation of Complex Loci in Primary Smooth Muscle Cells

In addition to lentiviral vectors, we designed a novel adenoviral vector (serotype 5) which includes the components required for the activation of CARMN, miR-143 and miR-145 in primary smooth muscle cells (FIG. 6A, B).

### CONCLUSION

CRISPR activator (CRISPRa) technology was adopted to simultaneously activate the transcription of multiple noncoding RNAs by activating their promoter region. In order to apply this strategy in a clinical setting, viral vectors were exploited for efficient ex vivo gene transfer. This novel concept was applied to CARMN and MIR503HG, which shows that i) it is possible to simultaneously activate the expression of lncRNA transcripts and microRNAs by targeting their main promoter and that ii) it is possible to use this approach in primary cells. Importantly, this can be achieved in primary cells by using only one viral vector. This reveals the high versatility of this system which can be used to enhance the transcriptional activation of other noncoding loci (lncRNA and microRNAs) by only adding the specific sgRNA targeting the promoter of interest. Nonetheless, this opens possibilities for the translation of this approach to different clinical settings.

### REFERENCES

1 Statello, L., Guo, C. J., Chen, L. L. & Huarte, M. Gene regulation by long non-coding RNAs and its biological functions. Nat Rev Mol Cell Biol 22, 96-118, doi:10.1038/s41580-020-00315-9 (2021).
2 Sallam, T., Sandhu, J. & Tontonoz, P. Long Noncoding RNA Discovery in Cardiovascular Disease: Decoding Form to Function. Circ Res 122, 155-166, doi:10.1161/CIRCRESAHA.117.311802 (2018).
3 Vacante, F. et al. CARMN Loss Regulates Smooth Muscle Cells and Accelerates Atherosclerosis in Mice. Circ Res 128, 1258-1275, doi:10.1161/CIRCRESAHA.120.318688 (2021).
4 Monteiro, J. P. et al. MIR503HG Loss Promotes Endothelial-to-Mesenchymal Transition in Vascular Disease. Circ Res 128, 1173-1190, doi:10.1161/CIRCRESAHA.120.318124 (2021).
5 Sun, Q., Song, Y. J. & Prasanth, K. V. One locus with two roles: microRNA-independent functions of microRNA-host-gene locus-encoded long noncoding RNAs. Wiley Interdiscip Rev RNA 12, e1625, doi:10.1002/wrna.1625 (2021).
6 Guttman, M. & Rinn, J. L. Modular regulatory principles of large non-coding RNAs. Nature 482, 339-346, doi:10.1038/nature10887 (2012).
7 Kim, T. K., Hemberg, M. & Gray, J. M. Enhancer RNAs: a class of long noncoding RNAs synthesized at enhancers. Cold Spring Harb Perspect Biol 7, a018622, doi:10.1101/cshperspect.a018622 (2015).
8 Uszczynska-Ratajczak, B., Lagarde, J., Frankish, A., Guigo, R. & Johnson, R. Towards a complete map of the human long non-coding RNA transcriptome. Nat Rev Genet 19, 535-548, doi:10.1038/s41576-018-0017-y (2018).
9 Perry, R. B. & Ulitsky, I. Therapy based on functional RNA elements. Science 373, 623-624, doi:10.1126/science.abj7969 (2021).
10 Winkle, M., El-Daly, S. M., Fabbri, M. & Calin, G. A. Noncoding RNA therapeutics—challenges and potential solutions. Nat Rev Drug Discov 20, 629-651, doi:10.1038/s41573-021-00219-z (2021).
11 Liu, L. et al. The H19 long noncoding RNA is a novel negative regulator of cardiomyocyte hypertrophy. Cardiovasc Res 111, 56-65, doi:10.1093/cvr/cvw078 (2016).
12 Profumo, V. et al. LEADeR role of miR-205 host gene as long noncoding RNA in prostate basal cell differentiation. Nat Commun 10, 307, doi:10.1038/s41467-018-08153-2 (2019).
13 Sun, Q. et al. MIR100 host gene-encoded lncRNAs regulate cell cycle by modulating the interaction between HuR and its target mRNAs. Nucleic Acids Res 46, 10405-10416, doi:10.1093/nar/gky696 (2018).
14 van Rooij, E. et al. A family of microRNAs encoded by myosin genes governs myosin expression and muscle performance. Dev Cell 17, 662-673, doi:10.1016/j.devcel.2009.10.013 (2009).

1. A construct for use in a method of transcriptional activation of a complex genomic locus, the construct comprising a single guide RNA (sgRNA) binding a regulatory element of the said complex genomic locus and a deactivated Cas, wherein the construct is introduced into a cell containing said complex genomic locus in order to activate the regulatory element to transcribe multiple transcripts within said complex genomic locus.

2. The construct for use in the method according to claim 1, wherein the method is an in vitro, in vivo or ex vivo method.

3. The construct for use in the method according to claims 1 to 2, wherein the complex genomic locus comprises one or more noncoding gene(s), multi-transcript noncoding gene(s), or a combination thereof.

4. The construct for use in the method according to claim 3, wherein the noncoding gene(s) comprise long non-coding RNA, microRNA, pri-microRNA, siRNA, piRNA, snoRNA, snRNA, exRNA, scaRNA or a combination thereof.

5. The construct for use in the method according to claim 4, wherein the noncoding RNA comprises long non-coding RNA, microRNA and/or pri-microRNA, or a combination thereof.

6. The construct for use in the method according to any preceding claim, wherein the deactivated Cas is fused to one or more transcriptional activators.

7. The construct for use in the method according to any preceding claim, wherein the transcriptional activators fused to the deactivated Cas comprise VP64, p65 and Rta.

8. The construct for use in the method according to any preceding claim, wherein the deactivated Cas is deactivated Cas9.

9. The construct for use in the method according to any preceding claim, wherein the complex genomic locus further comprise one or more protein coding gene(s).

10. The construct for use in the method according to any preceding claim, wherein the complex genomic loci comprise CARMN/miR-143/miR-145 or MIR503HG/miR-424/miR-503.

11. The construct for use in the method according to claim 10, wherein the guide RNA is selected from SEQ ID NO: 31 to SEQ ID NO: 34.

12. The construct for use in the method according to any preceding claim, wherein the construct is a component of a vector.

13. The construct for use in the method according to claim 12, wherein the vector is a viral vector.

14. The construct for use in the method according to claim 13, wherein the viral vector is an adenoviral vector, a lentiviral vector, a retroviral vector, an adeno-associated viral vector, a baculoviral vector, a vaccinia viral vector or a herpes simplex viral vector.

15. The construct for use in the method according to any preceding claim, wherein the cell is a eukaryotic cell.

16. The construct for use in the method according to claim 15, wherein the eukaryotic cell is a mammalian cell.

17. A vector for transcriptional activation of a complex genomic locus, wherein the vector comprises (i) a single guide RNA targeting a regulatory element of the said complex genomic locus, (ii) a deactivated Cas and (iii) one or more regulatory elements for the expression of the said single guide RNA and the said deactivated Cas.

18. The vector according to claim 17, wherein the vector comprises (i) a single guide RNA targeting a regulatory element of the said complex genomic locus, (ii) a first regulatory element for the expression of the said single guide RNA, (iii) a deactivated Cas and (iv) a second regulatory element for the expression of the said deactivated Cas.

19. The vector according to claims 17 to 18, wherein the single guide RNA targets transcriptional activation of one or more noncoding gene(s), multi-transcript noncoding gene(s), or a combination thereof.

20. The vector according to claims 17 to 19, wherein the one or more noncoding gene(s) comprise microRNA, lncRNA, pri-microRNA, siRNA, piRNA, snoRNA, snRNA, exRNA, scaRNA or a combination thereof.

21. The vector according to claim 20, wherein the noncoding gene(s) comprise long non-coding RNA, microRNA and/or pri-microRNA, or a combination thereof.

22. The vector according to claims 17 to 21, wherein the first regulatory element and the second regulatory element are selected from U6 and EFS.

23. The vector according to claims 17 to 22, wherein the deactivated Cas is fused to one or more transcriptional activators.

24. The vector according to claim 23, wherein the transcriptional activators fused to the deactivated Cas comprise VP64, p65 and Rta

25. The vector according to claims 17 to 24, wherein the deactivated Cas is deactivated Cas9.

26. The vector according to claims 17 to 25 wherein the complex genomic locus comprises CARMN/miR-143/miR-145 or MIR503HG/miR-424/miR-503.

27. The construct according to claim 26, wherein the single guide RNA is selected from SEQ ID NO: 31 to SEQ ID NO: 34.

28. The vector according to claims 17 to 27 comprising a viral vector.

29. The vector according to claim 28 comprising an adenoviral vector, a lentiviral vector, a retroviral vector, an adeno-associated viral vector, a baculoviral vector, a vaccinia viral vector or a herpes simplex viral vector.

30. The vector according to claims 17 to 29 for use in the treatment of atherosclerosis or pulmonary arterial hypertension.

31. The construct for use in the method according to claims 1 to 16 or the vector according to claims 17 to 29 for use in the prevention and/or regulation of pathological vascular remodelling, wherein the vector is introduced to a vascular cell.

32. The construct or the vector for use according to claim 31, wherein the vascular cell is an endothelial cell, a smooth muscle cell, a fibroblast, or a combination thereof.

33. A cell comprising transcriptional activation of a complex genomic locus using the construct for use in the method according to claims 1 to 16, wherein the cell is transfected with a vector according to any of claims 17 to 29.

34. The cell according to claim 33, wherein the cell is a vascular cell.

35. The cell according to claim 34, wherein the vascular cell is an endothelial cell, a smooth muscle cell, a fibroblast or a combination thereof.


SEQUENCE LISTING (34 sequences)

SEQ ID NO: 1 - DNA, 20 bp, synthetic construct
  source 1..20: mol_type = other DNA
tgggaatggaaacccttggt

SEQ ID NO: 2 - DNA, 20 bp, synthetic construct
  source 1..20: mol_type = other DNA
aagctgcatgttcaaggtcg

SEQ ID NO: 3 - DNA, 21 bp, synthetic construct
  source 1..21: mol_type = other DNA
ctgtaacaagtgcacaggcaa

SEQ ID NO: 4 - DNA, 21 bp, synthetic construct
  source 1..21: mol_type = other DNA
ttctgttgggaggcttggaga

SEQ ID NO: 5 - DNA, 21 bp, synthetic construct
  source 1..21: mol_type = other DNA
ccaggaaaggcaagggcgcta

SEQ ID NO: 6 - DNA, 21 bp, synthetic construct
  source 1..21: mol_type = other DNA
tgggagcggatgtgggtgccg

SEQ ID NO: 7 - DNA, 21 bp, synthetic construct
  source 1..21: mol_type = other DNA
gctgtaacaagtgcacaggca

SEQ ID NO: 8 - DNA, 20 bp, synthetic construct
  source 1..20: mol_type = other DNA
gtgggctcacagttctgtct

SEQ ID NO: 9 - DNA, 21 bp, synthetic construct
  source 1..21: mol_type = other DNA
gatccagagtaggagggagcc

SEQ ID NO: 10 - DNA, 20 bp, synthetic construct
  source 1..20: mol_type = other DNA
ggcccttgaatctgcttgcc

SEQ ID NO: 11 - DNA, 21 bp, synthetic construct
  source 1..21: mol_type = other DNA
agcctggaagtggctggatgt

SEQ ID NO: 12 - DNA, 20 bp, synthetic construct
  source 1..20: mol_type = other DNA
aaacgcatgcctgatggtgt

SEQ ID NO: 13 - DNA, 20 bp, synthetic construct
  source 1..20: mol_type = other DNA
gctcccaaagcaggaagacc

SEQ ID NO: 14 - DNA, 21 bp, synthetic construct
  source 1..21: mol_type = other DNA
gcccaacctcacaaatcctct

SEQ ID NO: 15 - DNA, 20 bp, synthetic construct
  source 1..20: mol_type = other DNA
aatgcaggaggcatgggcca

SEQ ID NO: 16 - DNA, 22 bp, synthetic construct
  source 1..22: mol_type = other DNA
gccacttgagtcagtgatggtg

SEQ ID NO: 17 - DNA, 21 bp, synthetic construct
  source 1..21: mol_type = other DNA
aaaagtcagaggctgtgggac

SEQ ID NO: 18 - DNA, 19 bp, synthetic construct
  source 1..19: mol_type = other DNA
ttgcccacacaatgcccta

SEQ ID NO: 19 - DNA, 21 bp, synthetic construct
  source 1..21: mol_type = other DNA
tgcctcttcagctcatataag

SEQ ID NO: 20 - DNA, 21 bp, synthetic construct
  source 1..21: mol_type = other DNA
gctagcgcccttgcctttcct

SEQ ID NO: 21 - DNA, 20 bp, synthetic construct
  source 1..20: mol_type = other DNA
cgccatgctgatgtcagaga

SEQ ID NO: 22 - DNA, 18 bp, synthetic construct
  source 1..18: mol_type = other DNA
gttctgtctccgggctgc

SEQ ID NO: 23 - DNA, 20 bp, synthetic construct
  source 1..20: mol_type = other DNA
ttgccttgacattctcgatg

SEQ ID NO: 24 - DNA, 20 bp, synthetic construct
  source 1..20: mol_type = other DNA
atcgctgtgatcgtcacttg

SEQ ID NO: 25 - DNA, 22 bp, synthetic construct
  source 1..22: mol_type = other DNA
ccacaggacaacgaagaaaacc

SEQ ID NO: 26 - DNA, 22 bp, synthetic construct
  source 1..22: mol_type = other DNA
tactccttttccagtcctcccc

SEQ ID NO: 27 - DNA, 20 bp, synthetic construct
  source 1..20: mol_type = other DNA
gtggaaccccacacaggaaa

SEQ ID NO: 28 - DNA, 22 bp, synthetic construct
  source 1..22: mol_type = other DNA
ggacagttgcccatattaacgg

SEQ ID NO: 29 - DNA, 21 bp, synthetic construct
  source 1..21: mol_type = other DNA
ggacctgagctgtcgatttca

SEQ ID NO: 30 - DNA, 20 bp, synthetic construct
  source 1..20: mol_type = other DNA
gagggatggaggtggcttta

SEQ ID NO: 31 - DNA, 20 bp, synthetic construct
  source 1..20: mol_type = other DNA
aaagcaacattcagcaccgg

SEQ ID NO: 32 - DNA, 20 bp, synthetic construct
  source 1..20: mol_type = other DNA
gctcccagcccagagcagcc

SEQ ID NO: 33 - DNA, 20 bp, synthetic construct
  source 1..20: mol_type = other DNA
atgaagggggatcgatctgg

SEQ ID NO: 34 - DNA, 20 bp, synthetic construct
  source 1..20: mol_type = other DNA
tgtagggaagggggttgtgc